(19) United States
(12) Patent Application Publication
(10) Pub. No.: US 2007/0032393 A1
(43) Pub. Date: Feb. 8, 2007

(54) Rinse-off conditioner comprising non-pregelatinized starch and fatty acid system for improved properties

(75) Inventors: Rajesh Patel, Middlebury, CT (US); Robert Sabin, Newtown, CT (US); Rosa Paredes, Shelton, CT (US)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Conopco, Inc. d/b/a UNILEVER

(21) Appl. No.: 11/197,695

(22) Filed: Aug. 4, 2005

(51) Int. Cl. A61K 8/00

(52) U.S. Cl. 510/119

(57) ABSTRACT

The invention provides rinse-off wet skin compositions comprising structuring systems which in turn comprise (a) non-pre-gelatinized starch; and (b) fatty acid, and which systems provide enhanced properties. The fatty acid also needs to be added to the aqueous phase during preparation of the formulation.

### FIELD OF THE INVENTION

The present invention relates to wet skin treatment compositions designed for use during bathing to impart desirable properties to skin, maintain its health and protect it from environmental stress. These wet skin compositions are compositions with little or no surfactant designed to provide emollient/conditioner (generally applied after cleansing composition has been used) and to be rinsed from the body after use.

### BACKGROUND

Rinse off skin conditioners generally are known in the art (U.S. Pat. No. 5,578,299 to Starch; U.S. Pat. No. 5,888,492 to Starch).

Such compositions are generally characterized by having high levels of emollients and low levels of surfactant. Typically the formulations are applied in the shower after cleansing and rinsed off to achieve oil/emollient deposition. Most existing formulations rely on high emollient levels (e.g., greater than 25% by wt. of composition) to achieve deposition. Synthetic polymers (e.g., cross-linked acrylate/methacrylate polymers such as Carbopol®; cellulose) are typically used to obtain stability and/or increase viscosity.

Applicants have now found that use of specific non-pregelatinized starch in combination with fatty acid in such low surfactant, rinse-off conditioner compositions helps to improve deposition, and to structure the composition in order to achieve good deposition, viscosity and stability, all while maintaining good aesthetic.

U.S. Pat. No. 6,699,488 to Deckner et al. discloses rinsable skin conditioning compositions, but fails to teach or suggest the non-pre-gelatinized starch, fatty acid structuring system of the subject invention.

Similarly, WO/03066016 to P&G and U.S. 2004/0223992 to Clapp et al. disclose wet skin treatment or rinse off conditioning compositions, but fail to teach or disclose the structuring system of the invention.

U.S. Pat. No. 6,716,440 to Aronson et al.; and U.S. Pat. No. 6,645,511 to Aronson et al. also disclose wet skin compositions, but again fail to teach or disclose the structuring system of the subject invention.

In applicants co-pending U.S. Ser. No. 10/849,100, to Tsaur, now allowed; and U.S. Ser. No. 10/849,627, now U.S. Pat. No. 6,903,057 to Tsaur, applicants disclose compositions comprising non-pregelatinized starch, and which may comprise fatty acid. These compositions, however, are cleansing compositions which require surfactant (e.g., greater than 2%). All examples in these applications contain far greater amounts of surfactant. Compositions of the subject invention comprise less than 4%, preferably less than 3%, more preferably less than 2%, more preferably 1.9% or less by wt. surfactant. Even more preferably 1.5% by wt. or less and more preferably 1% by wt. or less surfactant is used. Further, the compositions of the subject invention foam far less (e.g., due to low surfactant/high oil formulation).

U.S. Pat. No. 6,248,338 to Muller discloses specific pregelatinized, cross-linked starches. Starches of the subject invention (whether “modified” or not) need not be pre-gelatinized and preferably are not. Muller also discloses levels of surfactant up to 70% (column 6, line 7) and compositions having a wide range of pH value (pH between 2.5 and 12).

### BRIEF DESCRIPTION OF THE INVENTION

The subject invention relates to specific low-surfactant, rinse off conditioner compositions having modified starch, fatty acid structuring system. Compositions of the invention preferably use non-pregelatinized starches, have pH of about 5.5 to 7.5, preferably 6.0 to 7.0; have surfactant level of less than 4%, have low foam value, and emollient level of 10 to 30% by wt.

More specifically, the rinse-off wet skin compositions of the invention comprise:
(1) 0 to 4% by wt, preferably 0.5 to 1.9% by wt. surfactant;
(2) 5 to 50%, preferably 10 to 40% oil/emollient or mixture of two or more oil/emollients;
(3) 0 to 30% of auxiliary benefit agent (e.g., skin benefit agent sensory modifier) other than oil/emollient of (2);
(4) structuring system for said oil and/or auxiliary benefit agent comprising;
   (a) 0.5 to 10% by wt. non-pregelatinized starch; and
   (b) 0.5 to 8% by wt. fatty acid,
   wherein said fatty acid is added to water phase during preparation rather than to emollient/oil phase; and
(5) balance water.

These and other aspects, features and advantages will become apparent to those of ordinary skill in the art from a reading of the following detailed description and the appended claims. For the avoidance of doubt, any feature of one aspect of the present invention may be utilized in any other aspect of the invention. It is noted that the examples given in the description below are intended to clarify the invention and are not intended to limit the invention to those examples per se. Other than in the experimental examples, or where otherwise indicated, all numbers expressing quantities of ingredients or reaction conditions used herein are to be understood as modified in all instances by the term “about”. Similarly, all percentages are weight/weight percentages of the total composition unless otherwise indicated. Numerical ranges expressed in the format “from x to y” are understood to include x and y. When for a specific feature multiple preferred ranges are described in the format “from x to y”, it is understood that all ranges combining the different endpoints are also contemplated. Where the term “comprising” is used in the specification or claims, it is not intended to exclude any terms, steps or features not specifically recited. All temperatures are in degrees Celsius (° C.) unless specified otherwise. All measurements are in SI units unless specified otherwise. All documents cited are—in relevant part—incorporated herein by reference.

### BRIEF DESCRIPTION OF THE FIGURES

FIG. 1 is a schematic showing the difference when fatty acid is added to aqueous phase (does not phase separate) after 4 weeks at 20° C. compared to when added in emollient phase (does separate).

FIG. 2 is both a schematic and figure showing the difference of using fatty acid to structure versus not using fatty acid (e.g., no structuring achieved and product spreads).

### DETAILED DESCRIPTION OF THE INVENTION

The present invention relates to low surfactant, rinse off conditioner compositions in which use of certain starches in combination with fatty acid (e.g., starch, fatty acid structuring system) was found to structure compositions so as to achieve good oil/emollient deposition, increase viscosity and increase stability. It is also critical to the invention that the fatty acid be added to the aqueous phase rather than emollient phase during preparation.

Product Form

The compositions of the present invention are liquid or semi-liquid, cream or mousse compositions intended for topical application to the skin. The product forms contemplated for purposes of defining the compositions and methods of the present invention are typically rinsable formulations, by which is meant he product is applied topically to the skin and then, subsequently (i.e., within minutes), rinsed away with water; or otherwise the product is wiped off using a substrate or other suitable removal means. However, it is contemplated that the subject compositions may be used as leave-on lotions as well without deviating from the spirit of the invention.

All elements of the present invention will be described in detail hereafter.

Compositions of the invention comprise (1) oil/emollient; (2) structuring system comprising non-pregelatinized starch and fatty acid; and (3) water. Compositions comprise up to 4% surfactant (although they may have none), preferably less than 3%, more preferably less than 2%, more preferably 1.9% and less, even more preferably 1.5% or less and even more preferably 1% by wt. or less. Compositions may also optionally comprise other skin benefit agent and conventional cosmetic or skin care ingredients.

Oil/Emollients

The oils/emollients of the invention are skin compatible oils which comprise 5% to 50% by wt., preferably 10% to 40% by wt. of the composition.

A skin compatible oil is defined here as an oil that is liquid at the temperature at which bathing is carried out and that is deemed safe for use in cosmetics, said oil being either inert to the skin or actually beneficial. The most useful skin compatible oils for the present invention include ester oils, hydrocarbon oils, and silicone oils.

Ester oils as the name implies have at least one ester group in the molecule. One type of common ester oil useful in the present invention are the fatty acid mono and polyesters such as cetyl octanoate, octyl isonanoanate, myristyl lactate, cetyl lactate, isopropyl myristate, myristyl myristate, isopropyl palmitate, isopropyl adipate, butyl stearate, decyl oleate, cholesterol isostearate, glycerol monostearate, glycerol distearate, glycerol tristearate, alkyl lactate, alkyl citrate and alkyl tartrate; sucrose ester, sorbitol ester, and the like.

A second type of useful esters oil is predominantly comprised of triglycerides and modified triglycerides. These include vegetable oils such as jojoba, soybean, canola, sunflower, safflower, rice bran, avocado, almond, olive, sesame, persic, castor, coconut, and mink oils. Synthetic triglycerides can also be employed provided they are liquid at room temperature. Modified triglycerides include materials such as ethoxylated and maleated triglyceride derivatives, provided they are liquids. Proprietary ester blends such as those sold by Finetex as Finsolv® are also suitable, as is ethylhexanoic acid glyceride.

A third type of ester oil is liquid polyester formed from the reaction of a dicarboxylic acid and a diol. An example of polyesters suitable for the present invention is the polyesters marketed by ExxonMobil under the trade name PURESYN ESTER®.

A second class of skin compatible oils suitable for the present invention is liquid hydrocarbons. These include linear and branched oils such as liquid paraffin, squalene, squalane, mineral oil, low viscosity synthetic hydrocarbons such as polyalphaolefin sold by ExxonMobil under the trade name of PureSyn PAO® and polybutene under the trade name PANALANE® or INDOPOL®. Light (low viscosity) highly branched hydrocarbon oils are also suitable.

Petrolatum is a unique hydrocarbon material and a useful component of the present invention. Since it is only partially comprised of a liquid fraction at room temperature, it may be present by itself as oil or alternatively it may function as a “structurant” when admixed with other skin compatible oils.

A third class of useful skin compatible oils is silicone based. They include linear and cyclic polydimethyl siloxane, organo functional silicones (alkyl and alkyl aryl), and amino silicones.

Auxiliary Benefit Agents

The composition of the invention can optionally contain a variety of auxiliary agents. These auxiliary agents may be: functional skin benefit agents; sensory modifiers; and miscellaneous ingredients such as essential oils, and preservatives.

Specifically the compositions may comprise 0 to 30% by wt., preferably 5 to 25% by wt. of such auxiliary agents.

Functional Skin Benefit Agents

These materials function to in some way improve the state of the skin and include the following:
a) humectants used to retain water in the skin such as glycerol, sorbitiol, glycols, polyols, urea and their mixtures;
b) lipid barrier repair agents that are useful for strengthening, and replenishing the stratum corneum's barrier lipids such as cholesterol, cholesterol esters, ceramides, and pseudoceramides;
c) additional occlusive agents used to hold water in the stratum corneum such as natural and synthetic waxes and polyethylene;
d) vitamins used to strengthen the skin such as vitamin A, B, and E and vitamin alkyl esters, including vitamin C alkyl esters;
e) anti-aging agents used to exfoliate and stimulate cell turnover such as a and β hydroxy acids, retinol, and retinol esters;
f) Sunscreens such block the suns harmful UV rays such as octyl methoxy cinnamate (Parsol MCX) and butyl methoxy benzoylmethane (Parsol 1789), ultra-fine TiO2, ZnO and their mixtures;
g) skin lightening agents used to increase the lightness on the skin such as niacinamide;
h) antimicrobial agents such as 2-hydroxy-4,2′,4′-trichlorodiphenylether (Triclosan or Ergasan DP300) and 3,4,4′-trichlorocarbanilide (TCC);
i) antioxidants used to reduce photodamage and premature damage due to excessive oxidation such as ascorby palmitate, Vitamin E acetate, butylated hydroxyanisole and; 2,6-ditertiarybutylpara-cresol;
j) insect repellants such as N,N-dimethy-m-toluamide, 3-(N-butyl-Nacetyl)-aminopropionic acid, ethyl ester and dipropyl isocinchomeronate,
   mixtures of any of the foregoing components.
   Sensory Modifiers

These materials improve the aesthetic properties of the formulation and can be mixed with the structured oil phase before adding it into the aqueous phase or can be added to the aqueous phase to form a solution or dispersion. Suitable sensory modifiers include:
a′) emollient oils and emollient waxes used to improve the feel of the composition after rubbing into the skin, including: silicone resins, natural and synthetic waxes such as carnauba, spermaceti, beeswax, lanolin and derivatives thereof; higher fatty acids and alcohol;
b′) skin conditioning polymers that can alter the wet and dry skin feel provided by the composition. Such polymers include non-ionic polymers such as polyethylene oxide, polyvinyl alcohol, polyvinyl pyrrollidone, anionic polymers such as polyaspartate, poly maleates and sulfonates, cationic polymers and their mixtures. Suitable cationic polymers include Guar hydroxypropyltrimonium chloride, Quaternium-19, -23, 40, -57, poly(dimethyidiallylammonium chloride), poly(dimethyl butenyl ammonium chloride)-, w-bis(triethanolammonium chloride), poly (dipropyldiallylammonium chloride), poly(methyl-beta propaniodiallylammonium chloride), poly(diallylpiperidinium chloride), poly(vinyl pyridinium chloride), quaternized poly(vinyl alcohol), quaternized poly(dimethylaminoethylmethacrylate), and water insoluble polymers especially useful to modify wet skin feel such as polybutene, polyisobutene, polyisoprene, polybutadiene, polyalphaolefin and polyesters; and mixtures thereof;
c′) perfumes used to provide in-use fragrance and lingering fragrance on skin;
d′) distributing agents (also called a wetting agents) used to help the wet-skin treatment composition spread easily and uniformly over the body and reduce drag such as alkyl betaines, nonionic surfactants, silicone surfactants, and high molecular weight polyethene oxide;
e′) Emulsifying and dispersing agents that can reduce interfacial especially useful during processing. Some exemplary materials include: alkyl glycosides, other nonionic, cationic, and zwitterionic surfactants;
f′) chemosensory used to provide pleasant sensations like cooling such menthol and its derivatives, and certain essential oils well known in the art; and
g′) cosmetic grade solid particles that are effective in affecting either tactile or visual appearance of the skin such as talc, TiO2, silica or mica; for visual effect, color cosmetic grade pigments such as Timiron® MP pigments or Timiron® Splendid interference pigments are particular useful.

The sensory modifier of g′ are actually part of the dispersion incorporated into the structured oil phase.

Miscellaneous Agents

The composition of the invention can also contain a various essential oils such as, jasmine, camphor, white cedar, bitter orange peel, ryu, turpentine, cinnamon, bergamot, citrus unshiu, calamus, pine, lavender, bay, clove, hiba, eucalyptus, lemon, starflower, thyme, peppermint, rose, sage, menthol, cineole, eugenol, citral, Citronella, borneol, linalool, geraniol, evening primrose, thymol, spirantol, pinene, limonene and terpenoid oils.

Further useful classes of materials are preservatives, chelating agents and antioxidants. These materials are especially important when triglyceride ester oil are employed. Suitable preservatives for the present composition include: dimethyloldimethylhydantoin (Glydant XL1000), parabens, sorbic acid etc. Suitable chelators include tetrasodium ethylenediaminetetraacetate (EDTA) sold under the trade name VERSENE® 100XL, and hydroxyethilidene diphosphonic acid sold under the trade name Dequest® 2010 or mixtures in an amount of 0.01 to 1%, preferably 0.01 to 0.05%. An example of an antioxidant is butylated hydroxytoluene (BHT). Chelating agents are useful in binding metal ions including Ca/Mg as well as transition metal ions.

Still other useful agents include organic solvents, such as ethanol; auxiliary thickeners, coloring agents, opacifiers and pearlizers such as zinc stearate, magnesium stearate, TiO₂, EGMS (ethylene glycol monostearate) or Lytron® 621 (Styrene/Acrylate copolymer); all of which are useful in enhancing the appearance or cosmetic properties of the product.

A preferred auxiliary agent are the skin benefit agents, particularly the humectants, such as glycerol, sorbitol, glycols, etc., Preferably, these are used in an amount 5 to 25%, preferably 10 to 25% of composition.

Of course, auxiliary components may be present as mixtures of auxiliary benefit agent components.

Fatty Acid/Starch Structuring System

The compositions of the invention must further comprise a structuring system comprising 0.5 to 10%, preferably 1 to 10% by wt. of a non-pregelatinized starch and 0.5 to 8%, preferably 1 to 8% by wt. of fatty acid.

Starch

The non-pregelatinized starches of the invention are high molecular weight polysaccharides derived from plants such as corn, waxy corn, topioca, potato, wheat or rice. Plants synthesize starch and accumulate it in small discrete particles, called starch granules, having a size in the range of 1 to 100 micrometers depending on the source of plant. Non modified starch granules are insoluble in water at a temperature below 40° C. Starch can work as a thickening or structuring agent only after the starch granules are dissolved or are highly swollen by water. This can be achieved either by heat or by physical or chemical modification of the starch granules.

The temperature that is required to dissolve or to fully swell the starch granules varies with the plant source or the modification, if any, of a particular starch. For non modified starch granules, in general, potato starch gelatinizes at a lower temperature (around 65° C.) than waxy maize starch (around 70° C.) which, in turn, gelatinizes at a lower temperature than regular corn starch (around 75° C.). The gelatinization temperature (a critical temperature above which the intermolecular hydrogen bonds holding the granule together are weaker and the granule undergoes a rapid irreversible swelling by water) of a starch granule can be dramatically reduced by physically or chemically modifying the starch granule to make them suitable for low temperature processing. For example, Pure-Gel® Starches from Grain Processing Corporation are chemically modified corn starch granules having a gelatinization temperature around 53° C. which is well below the gelatinization temperature of a non-modified corn starch, which is around 75° C. Such “modified” corn starches are particularly preferred.

Modified or non-modified starch granules with gelatinization temperature between 300 to 85° C., preferably 300 to 70° C. are most preferred as the thickening/structuring agent of this invention. These types of starch granules are easy to process. It can be handled as a concentrate aqueous slurry (30 to 60% solids) which is flowable and pumpable at room temperature until the slurry is heated to a temperature above its gelatinization temperature.

In the subject invention, swelling or dissolution of starch granules can be done either, with or without the presence of surfactants, at a temperature higher than the gelatinization temperature of the specific starch granule. Higher processing temperature, in general, produces liquid cleansers with higher viscosity or better suspension properties due to higher swelling or better solubilization of these starch granules. It is preferred to process the starch granule in the presence of surfactants, if any. In the presence of surfactants, these starch granules swell to form starch gel particles after being processed at a temperature higher than its gelatinization temperature to thicken, structure and stabilize the liquid cleanser composition of this invention. Due to the way the liquid cleanser is stabilized by the swollen starch gel particles, the liquid cleanser of this invention has very shear thinning rheology, non stringy, non lumpy smooth appearance, and is easy to disperse in water during the use of the product.

In general, whatever starch is used, it is preferred that the starch granule, upon use in the final composition, swell at least 200% by volume, preferably at least 400%, more preferably at least 600%, and most preferably at least 800% by volume to form swollen starch gel particles with size in the range of 2 to 300 micrometers.

Examples of modified or non modified starch granules which require heat to swell or to dissolve to thicken the liquid cleanser composition of the invention are PureGel B990, PureGel B992, PureGel B980 or PureDent starches from Grain Processing. Examples of other commercially available starches granules are National 1545, Amioca corn starch, Structure Soaln (a modified potato starch), Clearjel, Hi Flo, National 1333, Colflo 67, National Frige, Novation 1600, Novation 2700 or Purity 420 from National Starch and Chemical Company. Chemically modified starch granules are preferred. Especially, starch granules modified with nonionic hydrophilic groups such as hydroxylethyl or hydroxypropyl and/or ionic groups such as phosphate, carboxylate, sulfate, sulfonate and dialkyl/trialkyl amino or quaternary ammonium ion are highly preferred. Aqueous solution of starches, especially those containing amylose molecules, tend to form aggregates with lumpy appearance during aging of the product. The stability problem of starch containing aqueous solutions can be prevented or minimized by modification of starch granules with nonionic and/or ionic hydrophilic groups. Other than better stability, the gelatinization temperature of the starch can be reduced dramatically by the level of hydrophilic groups attached to the starch molecules. In general, the gelatinization temperature decreases with increasing level of substitution. At high degree of substitution, the chemically modified starch granule becomes swellable in cold-water.

Modified starch granules, especially hydroxypropyl starch phosphate granules with gelatinization temperature in the range of 30 to 70° C., such as PureGel starches, are highly preferred.

Other than starch granules described above, there are pregelatinized cold water soluble starches which disperse and dissolve easily in cold water without the need of heating. These cold water soluble starches have been gelatinized and dried, so they will disperse and swell in cold water. These pregelatinized starches are not intended to be the type of starches covered by the invention. Examples of such pregelatinized cold water soluble starches, different from the non-pregelatinized starches of the subject invention, are Ultra-Sperse tapioca or waxy maize starch, Stir-N-set tapioca starch, National 5717 pregelatinized modified waxy maize starch, National 1215 pregelatinized unmodified corn starch, Structure ZEA, a hydroxypropyl modified corn starch or Structure XL, a cross-linked pregelatinized hydroxypropyl starch phosphate. All the starches mentioned above are commercially available from National Starch and Chemical Company.

Fatty Acid

The fatty acid can be C8 to C24 linear and/or branched, saturated and/or unsaturated fatty acid. Examples of such include lauric acid, oleic acid and myristic acid.

It is an important aspect of the invention that the fatty acid is added to the aqueous phase of the composition during preparation rather than to the emollient/oil phase. Without wishing to be bound by theory, when fatty acid is an aqueous phase, it does not phase separate (it does separate if in oil phase). Also it is more viscous (that is, when fatty acid is in oil phase, the composition is less viscous).

In general, the compositions are prepared by separately preparing aqueous phase (comprising, for example, glycerol, starch, fatty acid, thickeners, if any) and emollient phase (e.g., soybean, petrolatum).

If fatty acid is added to emollient phase this may effect the overall stability of final composition. In general, compositions of the invention should remain phase stable (no phase separation) for at least 4 weeks when left at temperature of 20° C.

Finally, the balance of the composition comprises water.

### EXAMPLES

Preparation

A typical composition of the invention was prepared as follows:

First an emollient phase A was prepared by using one emollient or adding a combination of emollients (e.g., 7.5% by wt. petrolatum and 7.5 soybean oil). The emollient(s) is heated to about 65° C.

An aqueous phase (phase B) is then prepared as follows:
(1) add thickener (e.g., cross-linked acrylate methacrylate polymer such as Carbopol®), if needed, ensuring to disperse as to minimize or remove all clays which may be present;
(2) add starch and heat to 80° C.;
(3) add glycerol and then fatty acid;
(4) slowly add pH adjuster (e.g., sodium hydroxide) to ensure pH of about 6 to 6.5;
(5) add surfactant (e.g., polydecyl glucoside);
(6) add whitening agents (e.g., TiO₂) and water.

Phase A and B are added and mixed for about 15 minutes. The pH is adjusted to about 6.3 to 6.5; temperature equilibrates at about 70° C. and batch is homogenized.

The batch is cooled to 60° C. and preservatives, fragrance and other minors, if any, added.

### Examples 1-7 and Comparatives A & B

The following Table uses examples of compositions of the invention

TABLE 5

| | INCI | Example | Example | Example | Compara- | Example | Example | Example | Example | Compara- |
|---|---|---|---|---|---|---|---|---|---|---|
| Tradename | Ingredient | 1 | 2 | 3 | tive A | 4 | 5 | 6 | 7 | tive B |
| Phase A | | | | | | | | | | |
| Petrolatum | Petrolatum | 20.0% | 15.0% | 7.5% | 7.5% | 7.5% | 12.5% | 12.5% | 20.0% | 20.0% |
| Silicone Gel | | | | | | | | 1.0% | | |
| (Y14898) | | | | | | | | | | |
| Soybean oil | Soybean oil | 0.0% | 0.0% | 7.5% | 7.5% | 7.5% | 7.5% | 7.5% | 0.0% | 0.0% |
| Fatty acid | — | — | — | — | 3.0% (OA) | — | — | — | — | — |
| Phase B | | | | | | | | | | |
| Glycerol | Glycerin | 20.0% | 15.00% | 14.4% | 14.4% | 14.4% | 15.0% | 15.0% | 20.0% | 20.0% |
| Starch | Pure Gel | 4.0% | 4.0% | 4.0% | 4.0% | 4.0% | 4.0% | 4.0% | 4.0% | 4.0% |
| | B990 | | | | | | | | | |
| | Ultrez 21 | | | | | | 0.15% | 0.15% | | |
| Fatty acid | Lauric acid | 3% (LA) | 3% (LA) | 3% (OA) | | 3% (OA) | 3% (OA) | 3% (OA) | 3% (OA) | 0% FA |
| Plantaren | Decyl | 0.950% | 1.000% | 0.950% | 0.950% | 0.950% | 0.950% | 1.750% | 0.950% | 0.950% |
| 2000N | glycoside | | | | | | | | | |
| Titanium | Titanium | 0.300% | 0.300% | 0.300% | 0.300% | 0.300% | 0.300% | 0.300% | 0.300% | 0.300% |
| dioxide | dioxide | | | | | | | | | |
| Dequest | EHDP | 0.051% | 0.051% | 0.051% | 0.051% | 0.051% | 0.051% | 0.051% | 0.051% | 0.051% |
| Versene | Tetrasodium | 0.020% | 0.020% | 0.020% | 0.020% | 0.020% | 0.020% | 0.020% | 0.020% | 0.020% |
| 100 XL | EDTA | | | | | | | | | |
| GlydantPlus | DMDM | 0.0% | 0.0% | 0.0% | 0.0% | 0.0% | 0.0% | 0.0% | 0.0% | 0.0% |
| | Hydantoin/ | | | | | | | | | |
| | Iodopropyl | | | | | | | | | |
| | Butyl | | | | | | | | | |
| | carbamate | | | | | | | | | |
| Fragrance | Fragrance | 0.0% | 0.0% | 0.4% | 0.4% | 0.4% | 0.4% | 0.4% | 0.0% | 0.0% |
| Comments | | High | High | No phase | OA added to | OA added | Excellent | Excellent | Structured | Not |
| | | viscosity | viscosity | separation | emollient | to water | deposition, | deposition, | and has | structured |
| | | | | observed, | phase, low | phase, high | stable, | stable, | excellent | and product |
| | | | | thicker | viscosity, | viscosity, | thick | thick | rheology; | spreads |
| | | | | product | phase | stable after | | | maintains | after |
| | | | | | separation | 1 month RT | | | structure | dispensing |
| | | | | | after 3 | | | | after | |
| | | | | | wks RT | | | | dispensing | |
| Deposition | | Excellent | Good | | — | Excellent | Excellent | Excellent | | |
| | | deposition | deposition | | | deposition | deposition | deposition | | |

LA—lauric acid
OA—Oleic acid
Carbopol is an acrylate/methacrylate thickening polymer

Several observations can be made from Table 5.

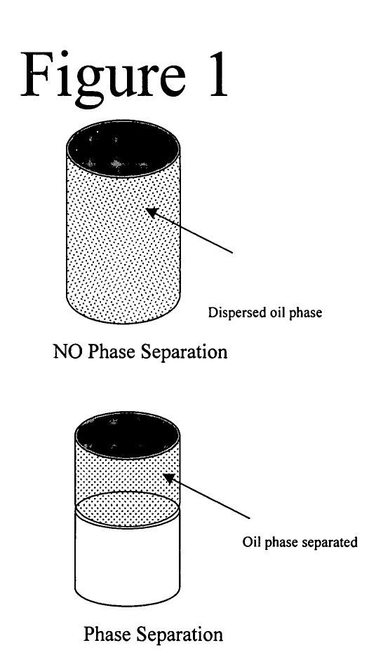

First in comparing Example 3 (fatty acid added in aqueous phase) and Comparative A (fatty acid added in emollient phase), it can be seen from comments, as well as from FIG. 1 (schematic), that, where FA is added to aqueous phase, there was no phase separation after 4 weeks (measured at 20° C.) and, when added at emollient phases there was phase separation. FIG. 1 shows that Comparative A composition was low viscosity and was “runny” and easily spread. By contrast, Example 3 was thick and not “runny”.

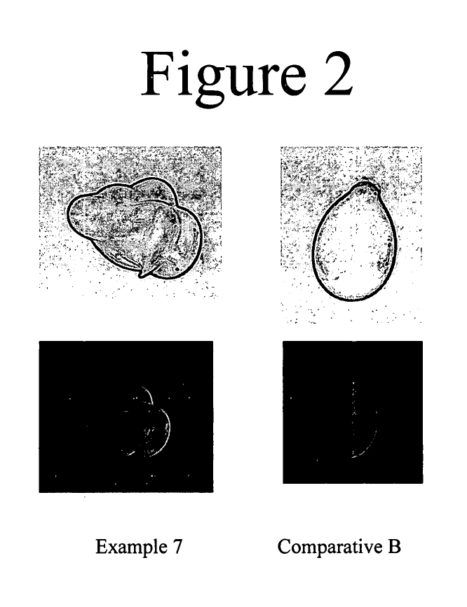

Comparing Example 7 to Comparative B, it can be seen that, when FA is used (Example 7), the composition is structured and maintains excellent rheology. When FA is not used (Comparative B), the product is not structured and spreads after dispersing. This can again be seen in schematic FIG. 2.

1. Rinse-off wet skin composition comprising:
(a) 0 to 4% by wt. surfactant;
(b) 5 to 50% by wt. oil/emollient;
(c) 0 to 30% by wt. auxiliary benefit agent;
(d) structuring system comprising:
(i) 0.5 to 10% non-pregelatinized starch; and
(ii) 0.5 to 8% fatty acid;
wherein said fatty acid is added to aqueous phase during preparation; and
(e) balance water.

2. A composition according to claim 1, comprising 0.1 to 3% by wt. surfactant.

3. A composition according to claim 1, comprising less than 2% by wt. surfactant.

4. A composition according to claim 1, wherein emollient is selected from the group consisting of petrolatum, soybean oil and mixtures thereof.
